(19) United States
(12) Patent Application Publication
(10) Pub. No.: US 2025/0041550 A1
(43) Pub. Date: Feb. 6, 2025

(54) CONNECTION PIECE FOR A MEDICAL VENTILATOR ASSEMBLY, A VENTILATOR SYSTEM, AND A METHOD OF MANUFACTURING A CONNECTION PIECE

(71) Applicant: Sentec AG, Therwil (CH)

(72) Inventors: Shawn Adam GOUGHNOUR, Coeur D'Alene, ID (US); Chad Thomas NUXOLL, Sandpoint, ID (US)

(73) Assignee: Sentec AG, Therwil, BL (CH)

(21) Appl. No.: 18/362,408

(22) Filed: Jul. 31, 2023

(51) Int. Cl. A61M 16/08; A61M 16/00; A61M 16/20

(52) U.S. Cl. CPC A61M 16/0875; A61M 16/0006; A61M 16/201

(57) ABSTRACT

A connection piece for a medical ventilator assembly which comprises a main body with a first inflow end and a second inflow end, and an outflow end. The first inflow end and the outflow end define a channel with a longitudinal axis. The second inflow end comprises a one-way valve adapted to allow gas flow toward the channel. The connection piece further comprises a leak valve arranged downstream of the one-way valve. The second inflow end is shaped and/or oriented with respect to the channel such that a gas flow from the second inflow end enters the channel, at an entry point, at an angle of less than 90° with respect to a direction of the longitudinal axis at said entry point from the first inflow end to the outflow end.

The present invention relates to a connection piece for a medical ventilator, a ventilator system and a method of manufacturing a connection piece according to the preamble of the independent claims.

Medical ventilators are used to support the breathing of patients by moving air in and out of the lungs. Ventilation may be necessary, for example, when a patient's spontaneous breathing is inadequate.

In some situations, usage of airway clearance therapy (ACT) as a mode or as an adjunct therapy may be desired in addition to a conventional ventilation mode. For example, intrapulmonary percussive ventilation (IPV), a type of ACT, may be used in combination with conventional ventilation.

Different ventilation modes may require dedicated ventilation devices which may need to be connected to a patient simultaneously. Therefore, connection pieces are known in the prior art which allow the connection of two or more ventilators such as to combine pressure and/or volume profiles to be delivered to the patient.

However, known connection pieces have several disadvantages. For example, known connection pieces may not be optimized for certain ventilation modes. In particular, known connection pieces may provide inadequate performance when used in combination with IPV and is some instances allow for connections which present a danger to the patient.

Thus, the object of the present invention is to overcome the drawbacks of the prior art, in particular to provide a connection piece which provides adequate performance in combination with IPV.

This and other objects are achieved by the connection piece according to the characterizing portion of the independent claim of the invention.

The invention is directed to a connection piece for a medical ventilator assembly. The connection piece comprises a main body having a first inflow end, a second inflow end, and an outflow end. The first inflow end and the outflow end define a channel which has a longitudinal axis. The second inflow end comprises a one-way valve. The one-way valve is adapted to allow gas flow toward the channel. The connection piece further comprises a leak valve arranged downstream of the one-way valve. The second inflow end is shaped and/or oriented with respect to the channel such that a gas flow from the second inflow end enters the channel, at an entry point, at an angle of less than 90° with respect to a direction of the longitudinal axis, from the first inflow end to the outflow end, at the entry point.

The connection piece is particularly suitable to connect an ACT device, for example an IPV device, to a conventional ventilator assembly. IPV devices providing pulsatile gas flow are known in the art.

Pulsatile gas, when entering the connection piece through the second inflow end, enters the channel at an angle of less than 90°, meaning that the pulsatile gas flow is generally oriented, at least partially, toward the outflow end of the connection piece. Therefore, the pulsatile gas may be transmitted into the breathing gas and to the patient more efficiently than in known devices. In particular, laminar flow may be retained, and turbulent flow reduced or avoided.

It will be understood that several shapes or orientations of the second inflow end is essential to achieve an airflow into the channel at an angle of less than 90°. The second inflow end may be configured as a straight cylindrical tube merging into the channel at an angle of less than 90°, but it is also conceivable to use a curved second inflow end which, e.g., which is oriented at an angle of less than 90° with respect to the channel only at an the entry point. Similarly, a baffle plate may be arranged in the channel to redirect a gas inflow from the second inflow end. In this case, the second inflow end may also be oriented at an angle of less than 90° with respect to the baffle face surface as this may result in a pulsatile gas flow entering the channel at an angle of less than 90°.

The leak valve is generally adapted to allow a portion of gas to escape the channel. The leak valve may be adjustable so that a user may increase or decrease the amount of gas which is allowed to leave the channel.

When an ACT device is used with a conventional/mechanical ventilation device simultaneously, in particular when the ACT device delivers percussive pulses, the total volume of the delivered gas is increased. The added flow may thus be reduced using the leak valve. In particular where the ACT device delivers IPV/pulsatile gas, the added flow may not be constant. In combination with conventional ventilation, the volume addition may cause pressure and/volume spikes which are outside of desired or even acceptable parameters. Therefore, the leak valve allows to combine pulsatile gas delivery (e.g. through IPV) simultaneously to conventional ventilation by reduction of the volume added into the breathing cycles of the conventional ventilator. As a result, volume control modes may also be accessible to the user even when ACT is additionally provided.

The second inflow end may be particularly adapted for being connected to a pulsatile breathing head and may have a round shape with an inner diameter of 22 mm. For example, standard receiving sockets for ventilation tubing are known in the art and commercially available.

In use, the first inflow end may be connected to a conventional ventilator machine as known in the art, typically via ventilator tube. For example, commercially available ventilators include Puritan Bennett™ 980 (Medtronic), Evita® series (Dräger), and G5 (Hamilton). The outflow end may be connected to further tubing and/or a patient interface to deliver breathing gas to the patient. The breathing gas may contain breathing cycles (from the conventional ventilator machine connected to the first inflow end) and/or high-frequency pulsatile gas (from an ACT device connected to the respective other inflow end).

The one-way valve may prevent breathing gas from being pushed out the second inflow end. Therefore, the connection piece enables the delivery of breathing cycles without the delivery of pulsatile gas flow (or any other form of ventilation) via the second inflow end. While different use cases of the connection piece are conceivable, this makes the connection piece according to the invention particularly suitable for medical treatments where the patient is continuously ventilated and intermittently receives ACT, e.g. IPV therapy. Breathing cycles may then be delivered through the channel, i.e., from the first inflow end toward the outflow end. If no pulsatile gas is delivered temporarily, the delivery of breathing cycles may lead to a pressure increase in the channel which may be sufficient to push gas back out via the second inflow end without a one-way valve. For the same reason, the connecting piece may be used in ventilation to retain the option of later IPV therapy.

Preferably, the second inflow end is arranged at an angle of less than 90° with respect to the direction to the longitudinal axis at the entry point from the first inflow end to the second inflow end.

Such an arrangement provides a particularly simple shape as the second inflow end may be configured as substantially straight cylindrical shape. Therefore, the connection piece may be particularly easy and cheap to produce and, due to the lower manufacturing complexity, less prone to manufacturing errors.

The leak valve may comprise a filter for filtering gas flowing from the channel to ambient through the leak valve, i.e. a leak gas flow.

Therefore, droplets and/or pathogens which may be exhaled by the patient may be filtered and prevented from reaching ambient air, providing improved safety for medical staff.

Preferably, the filter is held onto the leak valve by means of a filter cap.

For example, the leak valve may be formed by an opening in the connection piece which opens into the channel, and wherein a screw cap is adapted to close and open said opening. The filter may be placed on the opening and help by the screw cap.

Such an arrangement provides a particularly easy way to place, and therefore also replace, a filter and therefore keeps manufacturing cost low.

The leak valve may comprise a mechanism to adjust the leak gas flow, i.e., such as to increase or decrease the leak gas flow. Preferably, the mechanism also allows to stop the leak gas flow.

For example, if the leak valve comprises a screw cap as described above, the movement toward and away from an opening may decrease and increase, respectively, the amount of gas which is permitted to exit via the leak valve. Other mechanisms to adjust a leak gas flow are conceivable as well.

Particularly preferably, the leak valve comprises a leak valve cap. The leak valve cap may be screwed or screwable to the main body. The leak valve cap comprises a plug which is adapted for closing a leak opening in the main body when the leak valve cap is screwed on to the main body. The mechanism for adjusting the leak flow is a screwing mechanism for adjusting a distance between the plug and the leak opening.

Furthermore, a leak valve cap which is screwed or screwable on the main body is easy to replace.

The leak valve cap may, optionally, comprise a mechanical stop which prevents turning beyond a certain point. For example, a mechanical stop may prevent a user from accidentally screwing the leak valve cap off or to prevent complete closure of the leak valve. Additionally or alternatively, mechanical stops may be used to define an upper limit and/or a lower limit of allowable leak gas flow.

It will be understood that mechanical stops may prevent rotation beyond a certain degree and/or vertical movement.

The leak valve cap may be adapted to allow the leak flow through the leak opening which correlates, in particular linearly, with a rotational position of the leak valve cap. Optionally, the leak valve cap has at least one mechanical stop limiting a range of motion.

Such an arrangement is particularly advantageous in that it provides a flow regulation which is particularly easy and intuitive to adjust for an operator. It will be understood that the leak flow may increase and decrease in any increments suitable for a given application. The increment, i.e. the increase or decrease in leak flow per rotation may, in particular, be determined by a thread angle if the leak valve is screwable or screwed onto the connection piece.

Particularly preferably, the leak valve cap is configured such that a full rotation, i.e. 360°, brings the leak valve from fully closed to fully opened.

A filter cap may be attached to the leak valve cap, in particular by a first snap-on mechanism. The filter may be arranged between the leak valve cap and the filter cap.

Preferably, the one-way valve comprises a valve flapper.

A flapper valve may result in less resistance for the air flow in the desired flow direction (e.g. for the pulsatile gas flow) while still preventing flow in an opposite direction.

An opening of the one-way valve, when opened for gas flow, may have a size, in particular a preferably cross-sectional area, corresponding to at least half a size of a cross-sectional area of the second inflow end.

This may further reduce the resistance for the gas flow entering via the second inflow end and therefore increase the efficiency of the gas transmission and reduce turbulences.

The connection piece may comprise a cap which is attached or attachable to the main body by a tether. The cap may be adapted for closing the second inflow end. Particularly preferably, the cap is attached, via the tether, to the second inflow end.

When the second inflow end is not in use, e.g. because the patient is only ventilated with one ventilation device which is connected to the patient, it is advantageous to close the second inflow end when the ACT device (e.g. an IPV device) is not connected or providing therapy. For example, entry of unwanted substances (such as dirt, water, bodily fluids, etc.) into the second inflow end may be avoided.

The cap may comprise an anti-removal feature which hinders or prevents its accidental removal once installed.

It is also desirable to keep the cap attached to the connection piece to avoid e.g. misplacement.

The tether may have length sufficient to close another opening when not used to close the second inflow end. For example, in a typical use, a breathing head providing pulsatile gas flow into the second inflow end may be attached to the second inflow end. Such breathing heads typically comprise an exhalation port. The exhalation port may not be needed when breathing cycles (i.e. inhalation and exhalation) are provided by a conventional ventilator. Therefore, the exhalation port may be closed by the cap.

The cap may generally be adapted to fit on and close 22 mm-diameter ports. The cap may be configured to be mechanically flexible and/or stretchable and may close 22 mm inner-diameter or outer-diameter ports.

The tether may have a length of 2.5 cm to 10 cm, preferably about 6 cm. The tether and/or the cap may comprise or consist of silicone.

The second inflow end may be formed by a first duct portion and a second duct portion. The first duct portion may be integrally formed with at least one of the first inflow end and/or the outflow end. The second duct portion may be attached or attachable to the first duct portion, in particular by a second snap-on mechanism.

This provides for a particularly simple way of manufacturing the connection piece when further elements, e.g. the one-way valve and/or a cap tether are to be arranged in or with respect to the second inflow end.

The second snap-on mechanism may comprise a protrusion ring which prevents a torsion about a duct axis, i.e. a longitudinal axis of the duct, and/or decoupling of the first and second duct portion.

The protrusion may be arranged on either the first duct portion or the second duct portion. The respective other one of the first and second duct portion may have a matching opening, e.g. a slot, for the protrusion. When the first and second duct portions are connected, the protrusion may thus be arranged in the corresponding opening and prevent rotation around a duct axis.

The protrusion may be formed integrally with the first or the second duct portion and/or may be formed on a protrusion ring arranged on an outer surface of either duct portion.

The protrusion may prevent a rotation of the first duct portion with respect to the second duct portion.

It will be understood that more than one protrusion and/or more than one corresponding opening may be present on the first and/or second duct portion to provide additional safety and/or to allow for more than one rotational position of the first duct portion with respect to the second duct portion.

Preferably, the one-way valve is arranged substantially between the first and the second duct portion.

Preferably, the valve flapper is held against the first duction portion by the second duct portion.

The tether may be attached to the second inflow end by means of a ring arranged or arrangeable between the first and the second duct portion. The ring may be arranged, between the first and the second duct portions, on an outside surface of the second inflow end.

The one-way valve may comprise an opening which is offset with respect to the duct axis of the second inflow end.

Such an arrangement may result in improved sealing and opening of the one-way valve, in particular when the one-way valve is a flapper valve, because the flapper may be bulged toward a support structure and therefore pushed towards an outer sealing edge.

The invention is further directed to a ventilator system for airway clearance therapy and/or ventilation of a patient. The ventilation system comprises a source of pulsatile gas and a connection piece as described herein. The source of pulsatile gas is attached to the second inflow end of the connection piece. The source of pulsatile gas may be an IPV device.

Additionally or alternatively to a source of pulsatile gas, the ventilation system may comprise a conventional/mechanical ventilator which may be connected or connectable to the first inflow end of the connection piece.

Preferably, the pulsatile gas is delivered via a breathing head and the breathing head may be coupled directly, i.e. without any tubing in between, to the second inflow end.

Preferably, the breathing head has an expiratory port with a size which is substantially identical to size of the second inflow end.

A cap may be adapted for closing the second inflow end as well as the expiratory port of the breathing head. A tether attaching the cap to the connection piece may be long enough to reach the expiratory port of the breathing head when the breathing head is connected to the second inflow end.

Sources of pulsatile gas and breathing heads suitable for the ventilation system described herein are known in the art and commercially available.

For example, US 2021/299374 A1, which is incorporated herein by reference, describes a flow-regulated, time-cycled high-frequency percussive ventilator which is suitable to be used in a ventilator system according to the invention.

EP 3 646 912 A1, which is also incorporated herein by reference, discloses a breathing head for a percussive ventilation which may be used to deliver percussive pulses and may be connected to the second inflow end of the connection piece.

The invention is further directed to a method of manufacturing a connection piece. The connection piece may be a connection piece as described herein.

The method comprises providing a main body having a first inflow end and an outflow. The first inflow end and the second inflow end define a channel. The main body further comprises a second inflow end portion arranged at an angle of less than 90° in a direction of the channel. By means of a snap-on mechanism, a duct portion is attached to the second inflow end portion. At least one of a valve flapper and a ring is arranged between the duct portion and the second inflow end portion.

The ring may be connected or connectable to a tether and/or a cap.

The main body may further comprise a leak valve opening with a thread. The method may comprise a step of screwing a leak valve cap onto the thread.

The method may further comprise a step of arranging a filter on the leak valve cap, preferably by means of a filter cap. The filter cap may be snapped on the leak valve cap.

The method may further comprise arranging a protrusion between the duct portion and the second inflow end portion. The protrusion may be adapted to prevent a rotation, e.g. of the duct portion, about a duct axis, i.e. about a longitudinal axis of the second inflow end portion, and/or decoupling of the first and second duct portion.

The protrusion may prevent a rotation of the duct portion with respect to the second inflow end portion.

The protrusion may be formed integrally with the first or the second duct portion or may be formed on a separately formed protrusion ring.

The invention is further directed to a method of ventilating a patient. The method comprises using a first ventilating device which is not an airway clearance therapy device. The first ventilating device delivers breathing cycles to the first inflow end of a connection piece as described herein.

The second inflow end of the connection piece may be connected to a second ventilating device which is a airway clearance therapy device, preferably an IPV device. The second inflow end receives percussive pulses from the second ventilating device.

It will be understood that the ventilating devices of US 2021/299374 A1 and the breathing head of EP 3 646 912 A1 are suitable to be used in the method according to the invention to deliver percussive pulses.

Preferably, the second inflow end is connected, in particular, directly, to a breathing head through which the percussive pulses from the second ventilating device are received.

In the following, the invention is described in detail with reference to the following figures, showing:

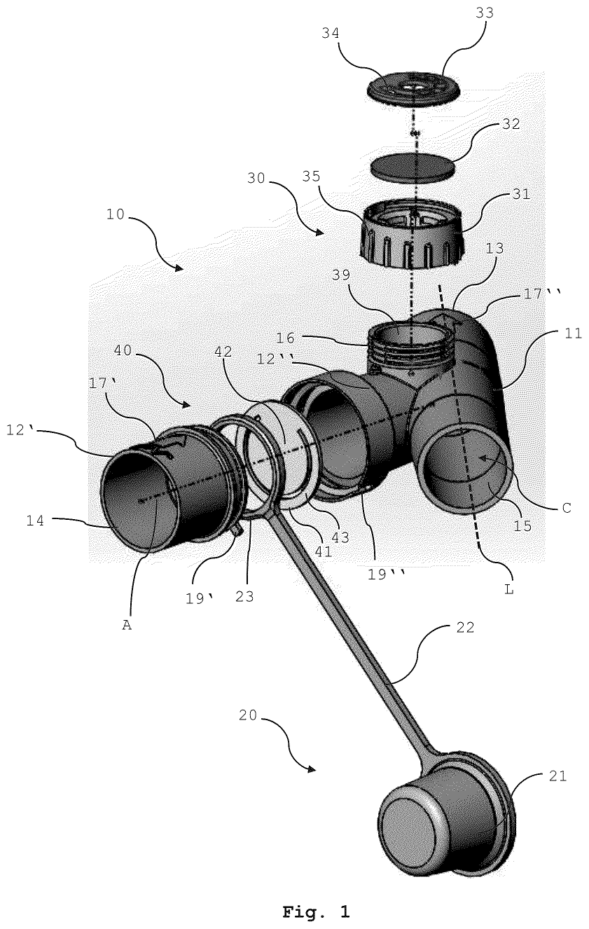

FIG. 1: an exploded view of a connection piece according to the invention.

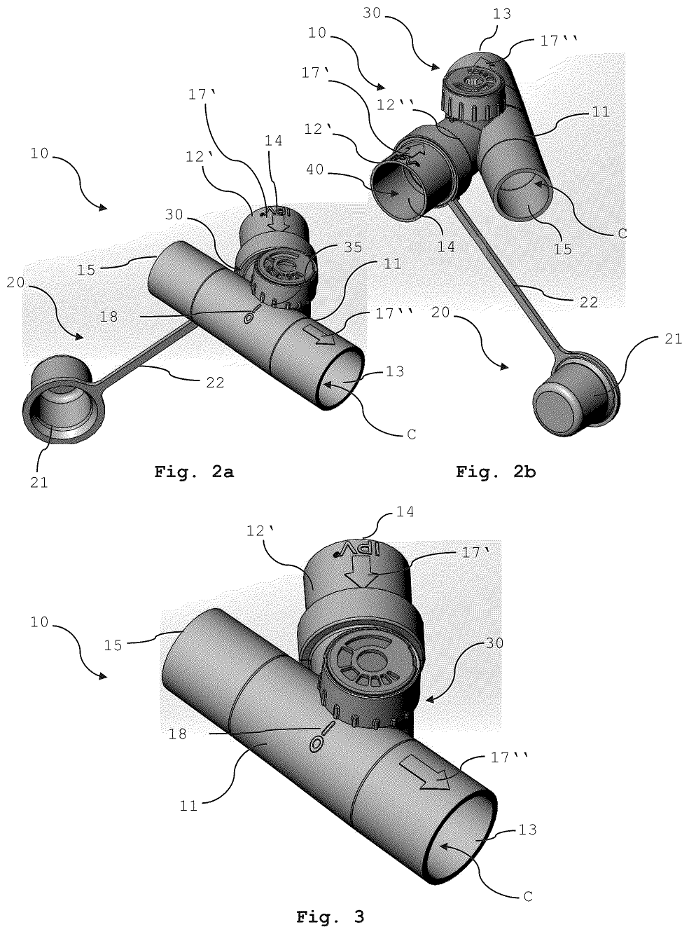

FIGS. 2a-2b: a first connection piece according to the invention in different perspective view.

FIG. 3: a perspective view of a second connection piece according to the invention.

FIG. 4: a cross-sectional view of the connection piece of FIG. 3.

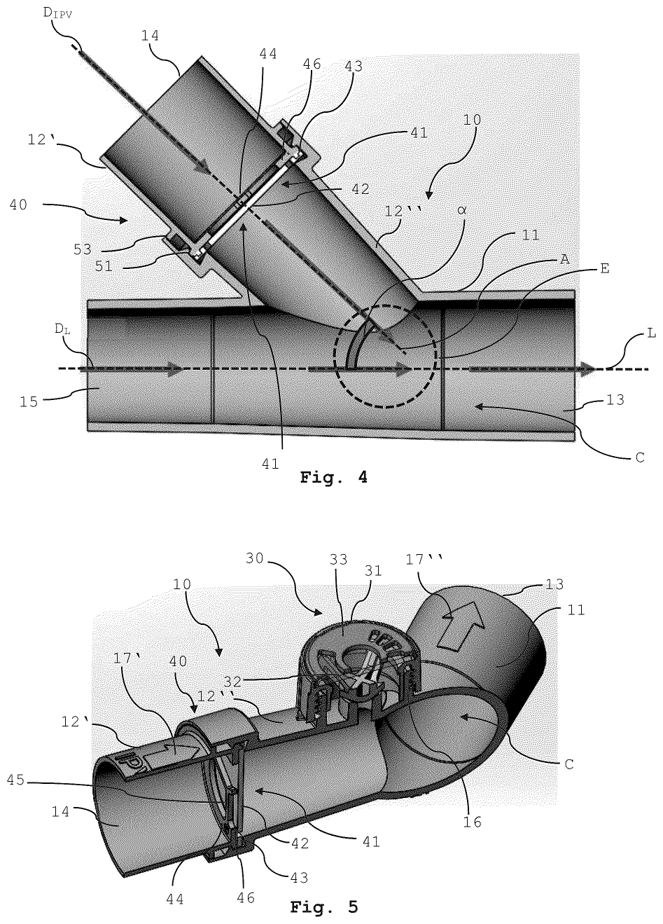

FIG. 5: a cutaway view of the connection piece of FIG. 3.

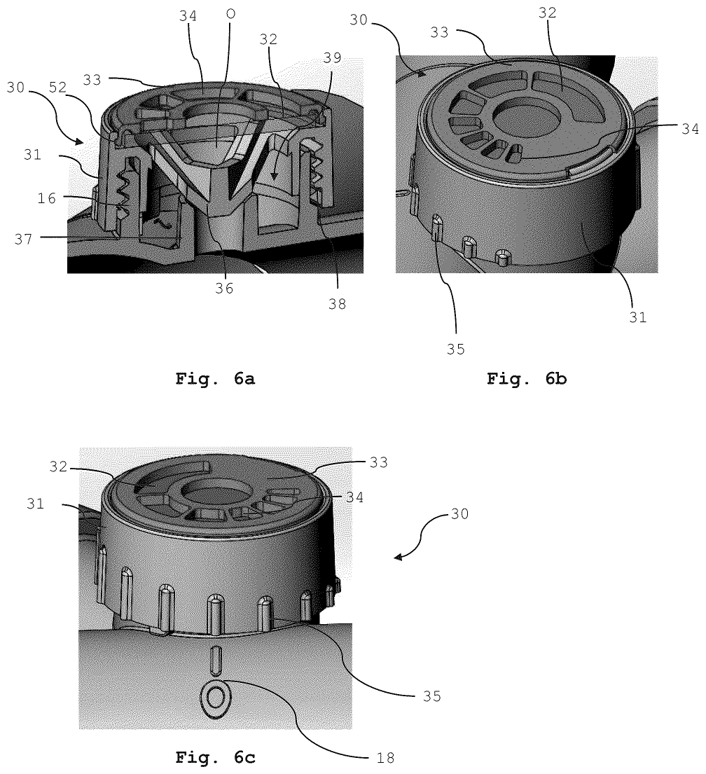

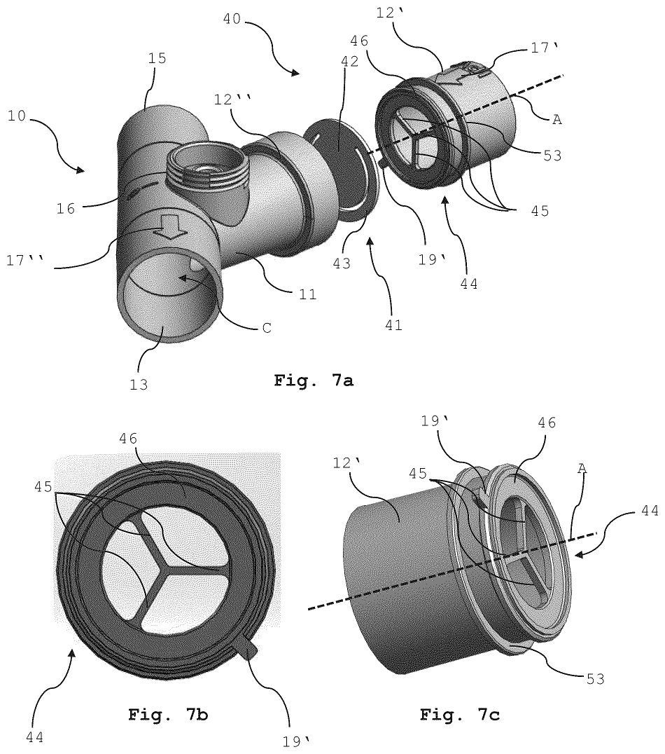

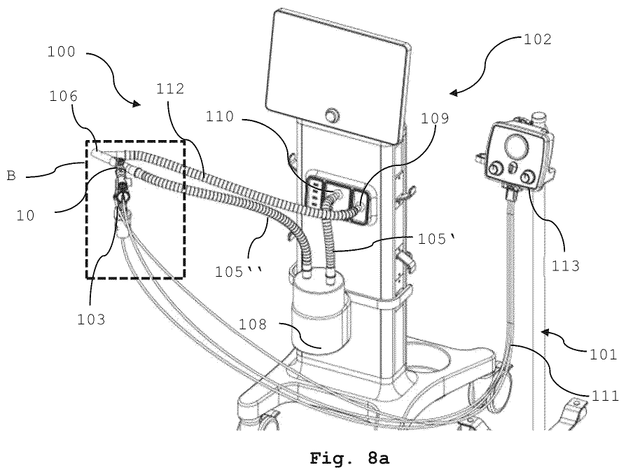

FIGS. 6a-6c: a detailed view of a leak valve.

FIGS. 7a-7c: a detailed view of a flapper valve.

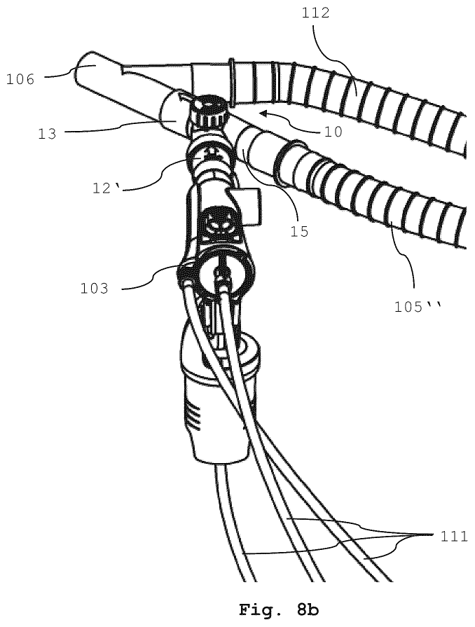

FIGS. 8a-8b: a ventilation system according to the invention.

FIG. 1 shows, in an exploded view, a connection piece 10 according to the invention. The connection piece is formed by a main body 11 having a first duct portion 12″, on which a second duct portion 12′ is snapped on. The second duct portion 12′ has an inner diameter of 22 mm and a depth of 21.5 mm. The main body 11 comprises a first inflow end 15 and an outflow end 13, both of which define a channel C with a longitudinal axis L. A marking 17″ on the outflow end indicates a flow direction of inhalation gas and therefore facilitates setting up a ventilation system for an operator. A second marking 17″ on the duct portion 12′ indicates an inflow direction for an IPV device (not shown). The main body 11 has a generally Y-shaped form, wherein a second branch of the Y-shape, forming the duct portion 12″, is connected to the duct portion 12′ and forming the second inflow end 14. A protrusion 19′ is formed on the duct portion 12′ and, when connected to the main body 11, is placed in a corresponding slot opening 19″ of the main body 11. Therefore, the duct portion 12′, when connected to the main body 11, is locked in a rotational position with respect to a duct axis A due to the protrusion 19′ and slot opening 19″. A one-way valve 40 is arranged in the second inflow end 14. The valve 40 is formed by a flapper 41 and a support surface (not visible). The flapper 41 is formed by a main flapper surface 42 surrounded by a flapper ring 43 on which the flapper main surface 42 is attached but free to hinge. The one-way valve is shown in more detail on FIGS. 7a-7c. In a direction along the duct axis A and toward the channel C, the flapper 41 is free to move and therefore allows gas to flow toward the channel C. The flapper 41 cannot move in the opposite direction due to the support surface and therefore acts as one-way valve (see FIGS. 4, 5 and 7a-7c). On the main body 11, at a downstream position of the one-way valve 40, a leak valve 30 is arranged. The leak valve 40 is formed by an opening 39 in the main body 11, which may be opened or closed by means of a leak valve cap 31 which is screwed on a thread 16 on opening 39. The leak valve 30 and its functionality is shown in more detail in FIGS. 6a-6c. The leak valve 40 further comprises a filter 32 arranged on the leak valve cap 31 and secured by a filter cap 33. The filter 32 is a commercially available bacterial viral hydrophobic filter. Therefore, a leak gas flow which escapes the connection piece 10 is filtered in order to provide additional safety, e.g. for medical staff. The leak valve cap 31 has markings 35 indicating qualitatively the amount of leak gas flow which is allowed by the leak valve 40. A similar marking 34 is contained on the filter cap 33. Both markings are shown in more detail on FIGS. 6a-6c. A closing mechanism 20 is adapted to close the second inflow end 14. The closing mechanism 20 comprises a cap 21 which is attached to the second inflow end 14 via a tether 22. The tether 22 is attached to a ring 23 arranged between the first duct portion 12′ and the second duct portion 12″.

FIG. 2a shows the connection piece 10 of FIG. 1 in an assembled view. For clarity, identical features will not be mentioned again but it will be understood that all the features mentioned in the context of FIG. 1 are also present in FIG. 2a. The connection piece 10 is made of polypropylene. Here, an index 18 arranged on the main body 11 and indicates, in conjunction with indices 35 on the leak valve cap 31, the qualitative amount of leak gas which is allowed to exit the leak valve 40. Rotation of the leak valve cap 31 opens and closes the leak valve 30 (see FIGS. 6a-6c) and places indices 35 of different lengths at the position of index 18, thus indicating to a user in a qualitative manner how much leak gas exits and also in which direction of rotation the leak gas flow is increased or decreased.

FIG. 2b shows the connection piece 10 of FIG. 2a in a different perspective view.

FIG. 3 shows a connection piece 10, in an assembled configuration, which is similar to the connection piece shown in FIGS. 2a-2b. Here, no tethered cap (see FIG. 2a, reference 20) is attached to the connection piece 10. For clarity, identical features are not repeated here, but the skilled person will understand that the all features and functionalities mentioned above, with the exception of the tethered cap, are present in the connection piece 10 shown here.

FIG. 4 shows a cross-section of a connection piece 10 in a plane defined by the duct axis A and the longitudinal axis L of the channel C. The duct portion 12′ is attached the duct portion 12″ of the main body 11 by means of a snap-on mechanism 51. An inflow direction D_{IPV} is shown along the duct axis A and corresponds to the flow direction of IPV pulses when the connection piece 10 is used with a pulsatile gas source (not shown). Along the longitudinal axis L, the direction of flow D_{L} of breathing gas from another ventilation source is shown. As shown here, an angle α at which the second inflow end 14 is arranged, and in which gas enters the channel via the second inflow end 14 in an entry point area E, is approximately 45°. The duct portion 12′ comprises a support surface 44 for the flapper 41 and a sealing ring 46, both of which are formed integrally with the duct portion 12′. The flapper main surface 42, when closed, sealingly engages outer ring 46 and therefore closes the one-way valve 40. The support surface 44 stops the flapper from being pushed against the flow direction D_{IPV}. The duct portion 12′, when snapped into main body 11, pushes and holds in place the outer ring 43 of the flapper 41. A protrusion ring 53 is arranged on an outer surface of the duct portion 12′ and stabilizes the duct portion 12′ in the respective other duct portion 12″, and by extension on the main body 11, by preventing bending away from the duct axis A.

FIG. 5 shows the connection piece 10 of FIG. 3 in a cutaway view. The cut plane corresponds to a plane which includes the duct axis A and is orthogonal to the cut section of FIG. 4. This cutaway view provides a more detailed view of the one-way valve 40 and in particular of the flapper support 44. The flapper support 44 comprises three arms 45 arranged in even angles in an opening defined by the outer ring portion 46 of the support 44. The flapper main surface 42 is free to move toward channel C, by hinging away from the outer ring of the flapper 43. However, the flapper support 44 prevents, however, a movement of flapper main surface 42 in the opposite direction. Here, the inner structure of the leak valve 30 is also visible but will be explained in more detail in FIGS. 6a-6c.

FIG. 6a shows a detailed sectional view of the leak valve 30. The leak valve cap 31 has an inner thread 38 which is in operable connection, when screwed in, with the outer thread 16 arranged with respect to the leak valve opening 39 of the main body 11. Inside the opening 39, the main body comprises an inner tube 37, having generally cylindrical shape with a cross-section which is smaller than the cross-sectional area of the opening 39. The tube 37 forms a fluid connection with an inside area of the main body 11, i.e. with the second inflow end 14 and/or with the channel C (see FIG. 5). The leak valve cap 31 comprises, a inner cone 36 arranged substantially along a center axis of the leak valve cap 31 and pointing toward the tube 37. When the leak valve 30 is closed, the inner cone 36 abuts the upper edge of tube 37 and therefore closes the fluid connection formed by the inner tube 37. The opening 39 is then closed and not in fluid connection with the channel or second inflow end. When the leak valve 30 is opened, by turning the leak valve cap 41 in a counterclockwise direction, the cone 36 is moved upward and away from the edge of the inner tube 37. Breathing gas, which may be air or air enriched with oxygen, can then escape between the cone 36 and inner tube 37. The flow of leak gas is regulated by the distance between the cone 36 and the inner tube 37, as any gas which exits leak valve cap 31 is allowed to exit the leak valve cap 31 via openings O therein. The leak gas flow exits the leak valve cap 31 through a filter 32 which is held by the filter cap 33. The filter cap is held on the leak valve cap 31 by a snap-on mechanism 52.

FIG. 6b shows the leak valve of FIG. 6a in a perspective view. The leak valve cap 31 has markings 35, on a circumferential outer wall, which have an increasing length in a clockwise direction. The markings 35 indicate, together with a marking on the main body 11 (see FIG. 6c, reference 18), qualitatively the amount of leak gas flow which is set. Turning the leak valve cap 31 in a counterclockwise direction moves longer markings 35 to marking 18 (see FIG. 6c) and thus indicates an increase in leak gas flow. Similarly, openings 34 in the filter cap 33 also indicate to an operator in which direction a leak gas flow may be increased or decreased. While openings 34 allow the leak gas to escape the leak valve 30 through the filter 32, the openings 34 are arranged around an outer area of the filter cap and with increasing sizes in a clockwise direction, therefore indicating an increase in leak gas flow when the leak valve 30 is turned in counterclockwise direction (and therefore moving larger openings 34 toward marking 18).

FIG. 6c shows the leak valve 30 of FIG. 6b from a different perspective wherein the marking 18 on the main body is visible which indicates, together with markings 35 and openings 34, the amount of leak gas flow exiting the leak valve 30. Markings 35 additionally provide grip for an operator to more easily turn the leak valve cap 31.

FIG. 7a shows an exploded view of the main body 11, duct portion 12′ and valve flapper 41 forming the one-way valve. The valve flapper 41, formed by main flapper surface 42 and outer ring 43, is arranged between the second duct portion 12′ and the first duct portion 12″ of the main body 11. When the duct portion 12′ and the main body 11 are snapped together, the outer ring 43 of the flapper 41 is held between the first and second duct portions 12′, 12″, whereas the flapper main surface 42 is hingedly movable towards the main body 11 so as to allow gas flow into the channel C. Arms 45, integrally formed as part of the duct 12′, form a support surface 44 for the flapper main surface 42. Protrusion 19′ is also formed integrally with the duct portion 12′ and is adapted in size and position to form an operable connection with a slot opening in main body (not visible, see FIG. 1).

FIG. 7b shows the support surface 44 of duct portion 12′ having arms 45, protrusion 19′, and outer support ring 46 and of support structure 44, in a view along the duct axis A (see FIG. 7a).

FIG. 7c shows the duct portion 12′ in a perspective view. The protrusion ring 53, which is offset along the duct axis A from the support surface 44, is visibly arranged on an outer surface of the duct portion 12′. The support surface 44, formed by arms 45, is offset along the duct axis A in a direction toward an inside of the duct portion 12′, i.e. in an opposite direction of the flow (see FIG. 4) across the one-way valve, with respect to a surface outer ring 46. Due to the bulged-in shape of the support surface 44, the flapper main surface 42 may bulge towards the support surface 44, when under pressure from gas in the channel C. The bulging provides an improved seal against outer support ring 46. Outer support ring 46 of the support surface 44 may be angled, with respect to the duct axis to exert a pushing force on the flapper main surface 42 in order to provide a bias toward closed position. A corresponding counter-shape in the main body may also be angled.

FIG. 8a shows a ventilation system 100 according to the invention. The ventilation system 100 is formed by a mechanical ventilator 102 and an airway clearance therapy (ACT) device 101. The ACT device 101 is formed by a source of pulsatile gas 113 which is in fluid connection with a breathing head 103 via tubing 111. As mentioned above, such ACT devices 101 are known in the art. The mechanical ventilator 102 has an expiration port 109 and an inspiration port 110. A first inspiratory tubing portion 105′, which is formed by a 22 mm tubing, is attached to inspiration port 110 and provides breathing cycles for a patient (not shown). The first tubing portion 105′ is attached humidifier 108. The breathing cycles which are transmitted from the inspiratory port 110 to the humidifier 108, and from there into second inspiratory tubing portion 105″ which leads to a connection piece 10 and further to a patient interface 106. The connection piece 10 may be any connection piece 10 described herein, as will be understood by the skilled person, and is not described in more detail here. Breathing head 103 is attached to a second inflow end of the connection piece 10 (see FIG. 8b) and receives percussive pulses from the source of pulsatile gas 113. The percussive pulses are transmitted into the gas flow, formed by breathing cycles, via connection piece 10. Therefore, the patient receives breathing cycles combined with percussive pulses for ACT. Expiratory cycles which are received by patient interface 106 are transmitted via expiratory tubing 112 into expiratory port 109 of mechanical ventilator 102.

As shown in FIG. 8a, percussive pulses are directly transmitted from the connection piece 10 into the patient interface 106. FIG. 8b shows a detailed view of panel B of FIG. 8a. As can be seen here, the breathing head 103 leads into the second inflow end 12′ of connection piece 10 and delivers percussive pulses into the connection piece 10. The second inspiratory tube portion 105″ is connected to the first inflow end 15 of connection piece 10. Breathing cycles and percussive pulses are thus delivered into connection piece 10, via first and second inflow ends, respectively, and exit connection piece 10 via outflow end 13. The outflow end 13 is connected to the patient interface 106, here without further tubing, though another tubing may be conceivable if desired e.g. due to space constraints.

1-24. (canceled)

25. A connection piece for a medical ventilator assembly, comprising a main body with a first inflow end and a second inflow end, and an outflow end, the first inflow end and the outflow end defining a channel with a longitudinal axis, wherein the second inflow end comprises a one-way valve adapted to allow gas flow toward the channel, the connection piece further comprising a leak valve arranged downstream of the one-way valve, wherein the second inflow end is shaped and/or oriented with respect to the channel such that a gas flow from the second inflow end enters the channel, at an entry point, at an angle of less than 90° with respect to a direction of the longitudinal axis at said entry point from the first inflow end to the outflow end.

26. The connection piece according to claim 25, wherein the second inflow end is arranged at an angle of less than 90° with respect to the direction of the longitudinal axis at said entry point from the first inflow end to the outflow end.

27. The connection piece according to claim 25, wherein the leak valve comprises a filter for filtering gas flowing from the channel to ambient through the leak valve.

28. The connection piece according to claim 26, wherein the filter is held onto the leak valve by means of a filter cap.

29. The connection piece according to claim 25, wherein the leak valve comprises a mechanism for adjusting a leak flow.

30. The connection piece according to claim 29, wherein the leak valve comprises a leak valve cap which is screwed or screwable to the main body, the leak valve cap comprising a plug adapted for closing a leak opening in the main body when the leak valve cap is screwed onto the main body, and wherein the mechanism for adjusting the leak flow is a screwing mechanism for adjusting a distance between the plug and the leak opening.

31. The connection piece according to claim 30, wherein the leak valve cap is adapted to allow the leak flow through the leak opening which correlates with a rotational position of the leak valve cap.

32. The connection piece according to claim 25, wherein the one-way valve comprises a valve flapper.

33. The connection piece according to claim 25, wherein an opening of the one-way valve, when opened for gas flow, has a size corresponding to at least half a size of a cross sectional area of the second inflow end.

34. The connection piece according to claim 25, further comprising a cap attached to the main body, by a tether and adapted for closing the second inflow end.

35. The connection piece according to claim 25, wherein the second inflow end is formed by a first duct portion integrally formed with at least one of the first inflow end and the outflow end, and a second duct portion attached or attachable to the first duct portion.

36. The connection piece according to claim 35, wherein the second snap-on mechanism comprises a protrusion which prevents a torsion about a duct axis and/or decoupling of the first and second duct portion.

37. The connection piece according to claim 25, wherein the one-way valve is arranged substantially between the first and the second duct portions.

38. The connection piece according to claim 25, wherein the one-way valve comprises an opening which is offset with respect to duct axis of the second inflow end.

39. A ventilator system for airway clearance therapy and/or ventilation of a patient, comprising a source of pulsatile gas and a connection piece according to claim 25, wherein the source of pulsatile gas is attached to the second inflow end.

40. A method of manufacturing a connection piece, comprising the steps of
providing a main body having a first inflow end and an outflow end defining a channel, and a second inflow end portion arranged at an angle of less than 90° in a direction of the channel,
attaching, by means of a snap-on mechanism, a duct portion to the second inflow end portion, wherein at least one of a valve flapper and a ring is arranged between the duct portion and the second inflow end portion.

41. The method according to claim 40, wherein the main body further comprises a leak valve opening with a thread, further comprising the step of screwing on a leak valve cap onto the thread.

42. The method according to claim 41, further comprising a step of arranging a filter on the leak valve cap.

43. The method according to claim 41, further comprising arranging a protrusion between the duct portion and the second inflow end portion, said protrusion being adapted to prevent a rotation about a duct axis and/or decoupling of duct portion and the second inflow end portion.

44. A method of ventilating a patient, using at least a first ventilating device which is not an airway clearance therapy device, and the first ventilation device delivers breathing cycles to the first inflow end of a connecting piece according to claim 25.

45. The method according to claim 44, wherein the second inflow end is connected to a second ventilating device, which is an airway clearance therapy device, and receives percussive pulses.

46. The connection piece according to claim 32, wherein the second inflow end is formed by a first duct portion integrally formed with at least one of the first inflow end and the outflow end, and a second duct portion attached or attachable to the first duct portion.

47. The connection piece according to claim 46, wherein the valve flapper is held against the first duct portion by the second duct portion.

48. The connection piece according to claim 28, wherein the leak valve comprises a leak valve cap which is screwed or screwable to the main body, the leak valve cap comprising a plug adapted for closing a leak opening in the main body when the leak valve cap is screwed onto the main body, and wherein the mechanism for adjusting the leak flow is a screwing mechanism for adjusting a distance between the plug and the leak opening.

49. The connection piece according to claim 48, wherein a filter cap is attached to the leak valve cap, and the filter is arranged between the leak valve cap and the filter cap.

50. The connection piece according to claim 34, wherein the second inflow end is formed by a first duct portion integrally formed with at least one of the first inflow end and the outflow end, and a second duct portion attached or attachable to the first duct portion.

51. The connection piece according to claim 50, wherein the tether is attached to the second inflow end by means of a ring arranged between the first duct portion and the second duct portion.
